(19) United States
(12) Patent Application Publication
(10) Pub. No.: US 2006/0084863 A1
(43) Pub. Date: Apr. 20, 2006

(54) Positional verification

(76) Inventors: Jacek Kluzik, Munich (DE); Martin Immerz, Munich (DE); Christoph Bodensteiner, Munich (DE); Christian Brack, Diedorf (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/249,728

(22) Filed: Oct. 13, 2005

Related U.S. Application Data
(60) Provisional application No. 60/622,414, filed on Oct. 27, 2004.

(30) Foreign Application Priority Data
Oct. 15, 2004 (EP) 04024599.5

(51) Int. Cl. A61B 5/05

(52) U.S. Cl. 600/426

(57) ABSTRACT

A method for detecting a change in position of at least one first marker or reference star relative to a first object, wherein the marker or reference star is attached to the first object. A position of at least one first characteristic point of the first object is ascertained via the first marker or reference star, and a position of at least one first characteristic point of a second object connected to the first object is ascertained. It is determined from the ascertained position of the at least two characteristic points whether the position of the at least one first marker or reference star has changed relative to the first object.

### RELATED APPLICATION DATA

This application claims priority of U.S. Provisional Application No. 60/622,414 filed on Oct. 27, 2004, which is incorporated herein by reference in its entirety.

### FIELD OF THE INVENTION

The present invention relates to position detection and, more particularly, to a method and device for recognizing a change in the position of at least one marker or reference star.

### BACKGROUND OF THE INVENTION

DE 694 32 961 T2 and DE 695 28 998 T2 describe arrays for determining the respective position of bodies. If a marker or array, such as a reference star, for example, is affixed to an object (e.g., a bone) by means of a single object or wire inserted into the bone (e.g., as described in DE 201 03 416 U1 belonging to the Applicant), then the reference star can be unintentionally twisted or moved during an operation (e.g., by being touched by a physician). Once twisted or moved, the reference star can no longer guarantee the precise determination of the body part or body structure position. In other words, a navigation or detection system for detecting the position of the body part or body structure may incorrectly ascertain the position of the body part or body structure due to the movement of the reference star.

One provision of the FDA states that during a computer-assisted navigated operation, a surgeon is to be reminded by a warning signal, approximately every 30 minutes, to verify that the markers affixed to bones or other body parts are correctly seated. Thus, for this verification, it is necessary to rely on the subjective assessment of the operating physician, prompted only at relatively large intervals in time.

### SUMMARY OF THE INVENTION

A marker or reference star (hereinafter referred to as a marker) can be attached to an object, such as a thigh bone (femur), which can be connected to a second object, such as a lower leg bone (tibia). Using characteristic points of the objects or bones, it is possible to ascertain from the position of characteristic points or axes whether the position of the at least one marker has changed relative to the object to which the marker is affixed.

At least one first marker (e.g., an active marker that emits infrared radiation or visible light, or a passive marker that reflects infrared radiation or visible light) can be attached or affixed to a first object, for example a first bone such as the shin bone, tibia, thigh bone or femur of a patient. The marker can be attached before or during a knee joint operation, for example. The position of the at least one first marker can be detected by a detection element, such as an IR (infrared) camera of a navigation system, for example, such that a spatial position of the at least one first marker is known or can be ascertained. If one object is registered, the position of at least one first characteristic point of the first object or bone (hereinafter referred to as object), such as a position of landmark points of the first object (e.g., characteristic points of the femur such as the condyles on the distal end of the femur, characteristic points of the tibia or shin bone such as the eminence on the tibial plateau, or characteristic points of the talus or ankle bone such as the middle of the talus) can be ascertained by means of the at least one first marker.

The position of at least one second characteristic point of a second object, such as the femur, tibia or talus, which is connected to the first object, preferably via a joint such as a knee joint, also can be determined. For example, a second marker can be attached to the second object such that a position of the at least one second characteristic point of the second object can be ascertained relative to the position of the first characteristic point of the first object. Generally speaking, the invention can be used with any object in which a positional relationship may have moved or has moved within pre-set limits. Preferably, the objects are or can be connected by a joint.

If the position of the first marker of the first object is changed (e.g., by offsetting or twisting the first marker) and/or the position of the second marker of the second object is changed (e.g., by offsetting or twisting the second marker), then the position of the first characteristic point of the first object and/or the second characteristic point of the second object relative to the respective markers also is changed. More specifically, the first and/or second characteristic point as detected by a detection unit or ascertained by a computational unit is changed such that the position of the characteristic point ascertained by means of the marker no longer matches the actual position on the object.

It is possible to ascertain from the ascertained position of the at least one first characteristic point of the first object and from the ascertained or known position of at least one second point of the second object whether the position of the at least one first marker has changed relative to the first object to which the at least one first marker or reference star is attached. The position of characteristic points of the first object relative to characteristic points of the second object can be determined and compared with anatomically possible relative positions, for example, in order to determine whether the detected positions are realistic, e.g., anatomically possible, or whether there is an unrealistic positional relationship between the objects. If the positional relationship is unrealistic, it may be established that the position of at least one reference star relative to an object has been twisted or offset. It is also possible to determine whether the position of the second marker or reference star has changed relative to the second object to which the second marker is affixed.

In particular, it is possible to determine the position of the at least two characteristic points relative to each other from the spatial position of the at least two characteristic points, and to determine from the ascertained position of the at least two characteristic points relative to each other whether the position of at least one marker has changed relative to the first object to which the first marker is attached or affixed, such that an alarm signal can be generated, for example.

A point or end point of the first object, such as landmark points of a bone (e.g., the femur, the tibia or the talus) can be used as the first characteristic point, for example. A point or end point of the second object, such as landmark points of the second bone (e.g., the thigh bone, the shin bone or the ankle joint bone or ankle bone) can be used as the second characteristic point, for example. Points, end points or foot points of an axis that describe the first object or of an axis that describes the second object, such as mechanical axes of the first or second object, for example, also can serve as characteristic points. These axes or straight lines can be connections between characteristic points or landmark points of an object, such as an axis or straight line through a condyle of the femur and the eminence on the tibial plateau, for example.

A distance between the at least two characteristic points (e.g., a distance between a first end point or foot point of a first axis that describes the first object such as a mechanical axis of the first object, and a second end point or foot point of a second axis that describes the second object such as a mechanical axis of the second object) can be ascertained. The ascertained distance of the at least two characteristic points can be compared with previously ascertained possible or admissible values for distances of the at least two characteristic points or with pre-set or stored anatomically possible distances. From this comparison, it is possible to determine whether the ascertained distance of the at least two characteristic points exceeds the pre-set, stored or previously ascertained maximum possible distance or value. In particular, it is possible to verify whether the ascertained distance of the at least two characteristic points is greater than the pre-set distance or value, wherein if the pre-set distance or value is exceeded, it can be deduced that the position of the at least one first marker has been changed or that the at least one first marker has been offset (e.g., to another point on the first object), or that the at least one first marker has been twisted or shifted about one of its axes (e.g., about the foot point of a device for attaching the at least one first marker as described in DE 201 03 416 U1).

In order to ascertain or verify the distance of the at least two characteristic points, one of the at least two characteristic points can be regarded as the center point of a sphere, wherein the radius of the sphere can be defined by a pre-set, stored or previously ascertained anatomically possible distance or value. Using the sphere, it is possible to deduce from a position of one characteristic point of the at least two characteristic points outside the sphere that the distance between the at least two characteristic points exceeds the pre-set maximum possible distance or value. If the maximum possible distance has been exceeded, then it can be concluded that the position of the at least one first marker has changed relative to the first object. The pre-set distance or value can be 0.1 mm up to 5 cm, for example, depending on what objects are being considered or on how the objects are connected. If, for example, the connection is a joint, such as a knee joint, then in order to determine the anatomically maximum possible distance of two end points of the bones adjacent to the joint, a shift in the position of the at least two characteristic points (e.g., due to the existing flexibility of ligaments or tendons which results in the bones being able to shift or twist with respect to each other) should be taken into account.

Preferably, the position of another characteristic point of the first object, such as the position of a landmark point of the first bone, and the position of another characteristic point of the second object, such as the position of a landmark point of the second bone, also can be detected or determined. The additional characteristic points can be further processed by a computational unit, for example, in order to examine whether the relative position of the axes assigned to the objects, as defined via the position of the points, is anatomically possible. In the computational unit, an axis of the first object, such as a mechanical axis of the first object or an axis or straight line through landmark points of the first object, can be ascertained from the at least two characteristic points of the first object. An axis of the second object, for example a mechanical axis of the second object or an axis or straight line through landmark points of the second object, can be determined from the at least two characteristic points of the second object, wherein the position of the axis of the first object relative to the position of the axis of the second object is examined or ascertained. It is possible to deduce from the position of the two axes relative to each other whether the position of the at least one first marker has changed or been changed relative to the first object to which the first marker is attached.

Preferably, the direction of the two axes can be spatially compared with pre-set directions or stored directions of two axes, with previously ascertained and stored directions, or anatomically possible relative positions of the axes of the first and second objects, for example. It is possible to deduce from this comparison whether the positions are outside an anatomically possible range, wherein it can be concluded that if the position of the two objects with respect to each other has not been changed (e.g., due to known positioning of the two objects relative to each other by a joint connecting both objects), a change in the position of the at least one first marker has taken place relative to the first object.

Preferably, it is also possible to ascertain an angle defined by the two object axes, for example the angle enclosed by the two object axes or bone axes at the intersection point of the object or bone axes, and to determine from the ascertained angle whether the position of the at least one first marker has changed relative to the first object on which the first marker is arranged. Preferable, the determination is made by comparing the ascertained angle with pre-set or known or previously ascertained and stored angle values. Further, the computational unit preferably verifies whether the ascertained angle or angle value is greater than a pre-set or previously ascertained, anatomically maximum possible angle value. If the pre-set or previously ascertained angle value, which can be stored as an absolute value or patient-specific value in a database or memory, is exceeded, the computational unit can recognize that a change in the position of the at least one first marker has taken place (e.g., the at least one first marker has been offset or turned about an axis).

If a change in the position of the at least one first marker is recognized using one of the methods described above, a warning signal can be output or generated that indicates a change in the position of the at least one first marker has taken place relative to the first object. This warning signal can be an acoustic or visual/optical signal, for example. The visual signal can indicate the position of the at least one first marker, for example. More particularly, the visual signal, via a display or computer or the like, can indicate the position of the at least one first marker relative to the first object to which the first marker is affixed, such that the surgeon can recognize that there has been an undesired change in the position of the at least one marker. It is possible to indicate on the screen via a numerical value or a graphical representation, for example, the change in position of the at least one marker (e.g., the relative shift, offset or twist of the marker), such that the surgeon can reverse or correct this change in position.

Advantageously, a marking can be attached to an object, for example, wherein the marking enables the marker to be easily repositioned without having to be re-registered (e.g., by overlapping a marking attached to a reference star holder with the object marking). The position of the at least one first marker can be verified at pre-set intervals that also can be regular or uniform, wherein this can be repeated every second or at an interval that can be between 1 ms and 10 s, for example.

The invention further provides a computer program which, when it is loaded onto a computer or is running on a computer, performs a method as described above. The invention further provides a program storage medium or a computer program product comprising such a program.

The invention also provides a device for recognizing a change in the position of at least one marker relative to an object to which the marker is affixed or attached, includes a first detection element, such as an infrared camera that can detect the position of at least one characteristic point of a first object (e.g., a landmark point of the first bone). Since the position of the at least one characteristic point relative to the marker attached to the first object is known once the object has been registered (or can be defined with respect to the at least one marker attached to the first object), changes in the position of the first object, such as movements of the first object, can thus be detected by the first detection element, wherein it is possible to deduce the three-dimensional position of the at least one characteristic point.

The device can further include a second detection element that can be the first detection element or can be a positioning element for fixedly positioning the second object. With the aid of the second detection element, it is possible to detect or ascertain the position of at least one characteristic point of a second object. Preferably, at least one marker can be arranged on the second object and can be detected by the detection element.

The device can further include a computational unit connected to the first detection element, wherein the computational unit ascertains the relative position of the at least two characteristic points from the spatial positions of the characteristic points detected by the detection element. By comparing the ascertained position with positions of the at least two characteristic points stored in a database or memory, the computational unit can determine from the ascertained relative position of the at least two characteristic points whether one of the markers has moved relative to the first object.

The device for recognizing a change in the position of at least one marker relative to the object to which the marker is affixed can also include a data output device, such as a warning device in the form of a screen or loudspeaker, which can be connected to the computational unit, and generates a warning signal when an undesired change in the position of the at least one marker is recognized.

### BRIEF DESCRIPTION OF THE DRAWINGS

FIG. 1 illustrates an exemplary device according to the present invention, in a first state.

FIG. 2 illustrates the device of FIG. 1, in a second state.

FIG. 3 is a block diagram of a computational unit that can be used to implement the method of the present invention.

### DETAILED DESCRIPTION

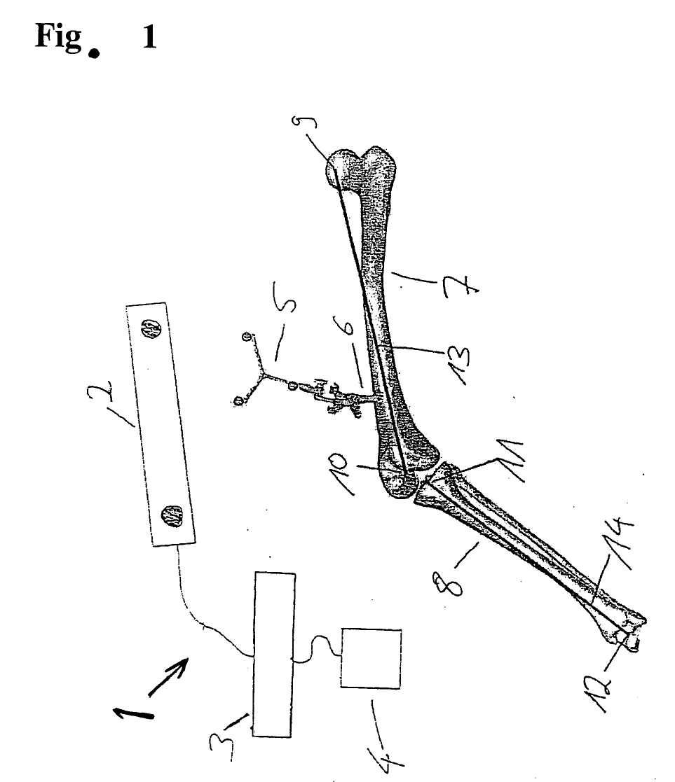

FIG. 1 shows a device 1 for verifying a position of an object, wherein a first object 7 is a thigh bone or femur 7, for example, and a second object 8 is a shin bone or tibia 8, for example. The device 1 includes an infrared camera 2 as a detection element, a computational unit 3 operatively coupled to the infrared camera 2, and a monitor or screen 4 as a data output device. A reference star 5 is attached to the thigh bone or femur 7, wherein the reference star 5 is preferably affixed to the femur 7 with the aid of an affixing device 6 in accordance with DE 201 03 416.6 U1, the contents of which is incorporated herein by reference in its entirety.

If, for example, the reference star 5 or the affixing device 6 for affixing the reference star 5 is touched or moved during an operation, the position of the reference star 5 can be changed relative to the femur 7 (e.g., if the device 6 is twisted or rotated such that an orientation of the reference star 5 relative to the femur 7 has been changed). In the first state shown in FIG. 1, two landmark or characteristic points 9 and 10 of the thigh bone 7 are registered with respect to the reference star 5, wherein the computational unit 3 calculates or virtually draws an axis 13 or straight line between the two characteristic points 9 and 10, as can be seen in FIG. 1. A second axis 14 is calculated or drawn by the computational unit 3 through two landmarks or characteristic points 11 and 12 of the shin bone 8, wherein the characteristic points 11 and 12 are registered with respect to another reference star (not shown) or by positioning the tibia 8 in a spatially fixed orientation.

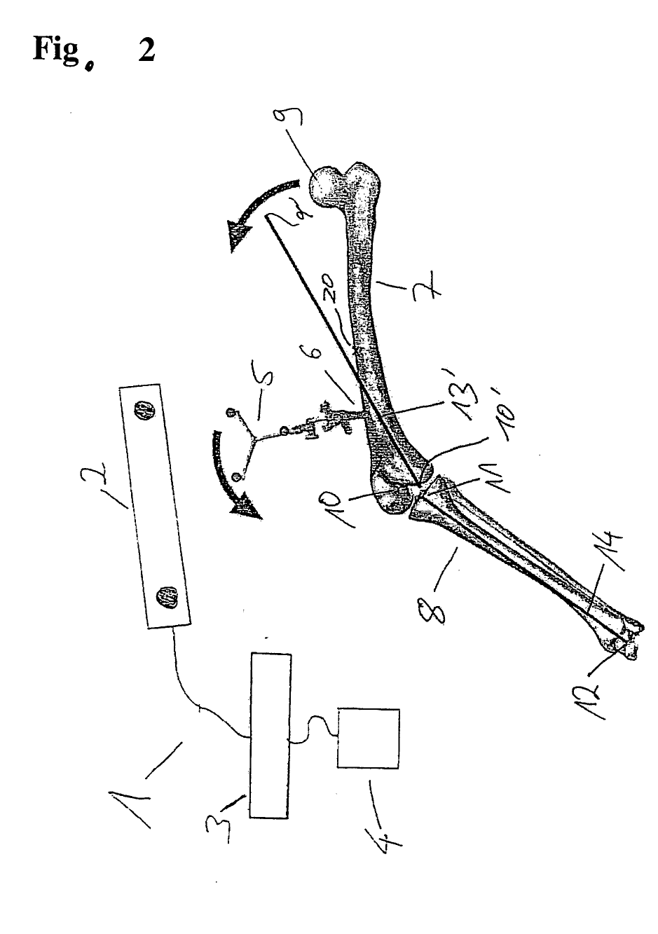

If, as shown in FIG. 2, the reference star 5 is unintentionally turned (e.g., by being touched by the surgeon), the computational unit 3, using the positions of the markers of the reference star 5 detected by the infrared camera 2, ascertains an incorrect position for the characteristic points 9′ and 10′. The incorrect position deviates from the actual position of the characteristic points 9 and 10 of the femur 7, whereby the ascertained axis 13′ of the femur 7 is shifted, as shown in FIG. 2. By comparing with previously stored anatomically possible relative positions of the mechanical femoral and tibial axes, the computational unit 3 recognizes that the relative position of the axis 13′ (which describes the thigh bone 7) and the axis 14 (which describes the shin bone 8) has moved outside an anatomically possible range. The computational unit 3 then deduces that the position of the reference star 5 has changed with respect to the thigh bone 7 or the reference star 5 has been twisted relative to the thigh bone 7. A monitor or screen 4, for example, operatively coupled to the computational unit 3 displays a warning signal for the physician that indicates the reference star 5 has moved or twisted or, for example, graphically indicates on the screen 4 the relative movement of the reference star 5 and/or the movement required to restore the reference star 5 to its registered position. Using a marking 20 on the femur 7, the reference star 5 also can be turned back to the initial position (e.g., its position prior to being disturbed or moved).

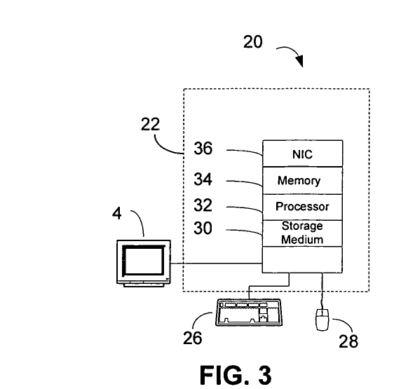

Moving to FIG. 3, a computational unit 3 for executing a computer program in accordance with the present invention is illustrated. The computational unit 3 includes a computer 22 for processing data, and a display 4, such as a CRT, LCD, or the like, for viewing system information. A keyboard 26 and pointing device 28 may be used for data entry, data display, screen navigation, etc. The keyboard 26 and pointing device 28 may be separate from the computer 22 or they may be integral to it. A computer mouse or other device that points to or otherwise identifies a location, action, etc., e.g., by a point and click method or some other method, are examples of a pointing device. Alternatively, a touch screen (not shown) may be used in place of the keyboard 26 and pointing device 28. A touch screen is well known by those skilled in the art and will not be described in detail herein. Briefly, a touch screen implements a thin transparent membrane over the viewing area of the display 4. Touching the viewing area sends a signal to the computer 22 indicative of the location touched on the screen. The computer 22 may equate the signal in a manner equivalent to a pointing device and act accordingly. For example, an object on the display 4 may be designated in software as having a particular function (e.g., view a different screen). Touching the object may have the same effect as directing the pointing device 28 over the object and selecting the object with the pointing device, e.g., by clicking a mouse. Touch screens may be beneficial when the available space for a keyboard 26 and/or a pointing device 28 is limited.

Included in the computer 22 is a storage medium 30 for storing information, such as application data, screen information, programs, etc. The storage medium 30 may be a hard drive, for example. A processor 32, such as an AMD Athlon 64™ processor or an Intel Pentium IV® processor, combined with a memory 34 and the storage medium 30 execute programs to perform various functions, such as data entry, numerical calculations, screen display, system setup, etc. A network interface card (NIC) 36 allows the computer 22 to communicate external devices.

The actual code for performing the functions described herein can be readily programmed by a person having ordinary skill in the art of computer programming in any of a number of conventional programming languages based on the disclosure herein. Consequently, further detail as to the particular code itself has been omitted for sake of brevity.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for detecting a change in position of at least one first marker or reference star relative to a first object, said marker or reference star being attached to said first object, comprising:
ascertaining a position of at least one first characteristic point of the first object via the first marker or reference star;
ascertaining a position of at least one first characteristic point of a second object connected to the first object; and
determining from the ascertained position of the at least two characteristic points whether the position of the at least one first marker or reference star has changed relative to the first object.

2. The method as set forth in claim 1, further comprising:
using an end point of the first object or an end point of an axis that describes the first object as the first characteristic point; and
using an end point of the second object or an end point of an axis that describes the second object as the second characteristic point.

3. The method as set forth in claim 1, wherein ascertaining a position of at least one first characteristic point of the first object includes ascertaining at least two characteristic points relative to each other, and further comprising determining from the ascertained position of the at least two characteristic points of the first object whether the position of the at least one first marker or reference star has changed relative to the first object.

4. The method as set forth in claim 3, wherein ascertaining at least two characteristic points of the first object includes ascertaining a distance between the at least two characteristic points and wherein
determining whether the position of the at least on marker or reference star has changed includes determining whether the ascertained distance of the at least two characteristic points of the first object exceeds a pre-set distance, and if the pre-set distance has been exceeded, concluding that the position of the at least one first marker or reference star has changed relative to the first object.

5. The method as set forth in claim 1, further comprising:
determining a second characteristic point of the first object, wherein the first and second characteristic points of the first object define a first axis; and
determining a second characteristic point of the second object, wherein the first and second characteristic point of the second object define a second axis; and
ascertaining from the position of the first axis relative to the position of the second axis whether the position of the at least one first marker or reference star has changed relative to the first object.

6. The method as set forth in claim 5, further comprising:
ascertaining an angle defined by the first and second axes, and
concluding the position of the at least one first marker or reference star has changed relative to the first object based on the ascertained angle.

7. The method of claim 6, wherein concluding includes comparing the ascertained angle to a pre-set angle, and if the ascertained angle exceeds the pre-set angle, concluding that the position of the first marker or reference star has changed relative to the first object.

8. The method as set forth claim 6, further comprising using a marking attached to the first object to return or restore the marker or reference star to an initial position.

9. The method as set forth in claim 1, further comprising generating a signal when the change in the position of the at least one first marker or reference star relative to the first object is determined.

10. The method as set forth in claim 9, wherein generating a signal includes generating a warning signal.

11. The method as set forth in claim 1, further comprising repeating the steps at pre-set intervals.

12. The method as set forth in claim 1, wherein repeating the steps includes using intervals between about 1 millisecond to about 10 seconds.

13. A computer program which, when it is running on a computer or is loaded onto a computer, performs the method as set forth in claim 1.

14. A program storage medium or computer program product comprising the computer program as set forth in claim 13.

15. A device for detecting a change in position of at least one marker or reference star, comprising.
a first detection element configured to determine a position of at least one characteristic point of a first object via a marker or reference star attached to the first object;
a second detection element configured to determine a position of at least one characteristic point of a second object; and
a computational unit operatively coupled to the first detection element, said computational unit configured to ascertain a relative position of the at least two characteristic points, wherein based on the relative position of the at least two characteristic points, the computational unit ascertains whether one of the markers or reference stars has moved relative to the first object.

16. The device as set forth in claim 15, wherein at least one marker or reference star is attached to the second object, and the positional relationship of the second object relative to the first object is known.

17. The device as set forth in claim 15, wherein the first detection element is an infrared camera.

18. The device as set forth in claim 15, wherein the second detection element is an infrared camera operatively coupled to the computational unit or is a positioning element.

19. The device as set forth in claim 15, further comprising a data output device operatively coupled to the computational unit for outputting a signal when the change in the position of the at least one marker or reference star relative to the object is detected.

20. The device as set forth in claim 19, wherein the device is a warning device and the signal is a warning signal.

21. A system for recognizing a change in position of at least one marker or reference star relative to an object, said marker or reference star being attached to said object, comprising a device as set forth in claim 15 and at least one marker or reference star.

22. A program embodied in a computer-readable medium for detecting a change in position of at least one first marker or reference star relative to a first object, said marker or reference star being attached to said first object, comprising:
code that ascertains a position of at least one first characteristic point of the first object via the first marker or reference star;
code that ascertains a position of at least one first characteristic point of a second object connected to the first object; and
code that determines from the ascertained position of the at least two characteristic points whether the position of the at least one first marker or reference star has changed relative to the first object.

23. A system for detecting a change in position of at least one first marker or reference star relative to a first object, said marker or reference star being attached to said first object, comprising:
a processor circuit having a processor and a memory;
an position detection sub-system stored in the memory and executable by the processor, the position detection sub-system comprising:
logic that ascertains a position of at least one first characteristic point of the first object via the first marker or reference star;
logic that ascertains a position of at least one first characteristic point of a second object connected to the first object; and
logic that determines from the ascertained position of the at least two characteristic points whether the position of the at least one first marker or reference star has changed relative to the first object.
